(19) United States
(12) Patent Application Publication
(10) Pub. No.: US 2024/0024163 A1
(43) Pub. Date: Jan. 25, 2024

(54) METHOD OF MANUFACTURING A FABRIC FOR A WOUND DRESSING

(71) Applicant: T.J.Smith and Nephew,Limited, Hull (GB)

(72) Inventors: Samuel John Mortimer, Kingston upon Hull (GB); Fraser George Weedon, Hull (GB); Amy Nicole Wheldrake, Bristol (GB)

(21) Appl. No.: 18/027,861

(22) PCT Filed: Sep. 21, 2021
(86) PCT No.: PCT/EP2021/075988

§ 371 Date: Mar. 22, 2023

(30) Foreign Application Priority Data
Sep. 22, 2020 (GB) 2014938.1

(51) Int. Cl. A61F 13/00; D04B 1/10; D04B 21/06; D04B 15/10; D04B 27/00; D04B 15/66

(52) U.S. Cl. CPC A61F 13/00995; D04B 1/10; D04B 21/06; D04B 15/10; D04B 27/00; D04B 15/66; D10B 2509/022; D10B 2401/02; D10B 2403/021; A61F 2013/00238

(57) ABSTRACT

The disclosed technology relates to methods of manufacturing a fabric (100) for a wound dressing in which a knitting machine (500) is configured to knit a plurality of yarns to form a knitted fabric (100), and a fabric formed according to the method. The disclosed technology also relates to a system, a computer program and a non-transitory computer readable medium.

### FIELD

The disclosed technology relates to methods of manufacturing a fabric for a wound dressing and a fabric formed according to the method. The disclosed technology also relates to a system, a computer program and a non-transitory computer readable medium.

### BACKGROUND

Many different types of wound dressings are known for aiding the healing process of a human or animal. These different types of wound dressing include many different types of materials and layers, for example, gauze, pads, foam pads or multi-layer wound dressings. Components used in wound dressings are typically produced using machines and subsequently cut to the required size. Although this approach may be acceptable, it can result in significant amounts of wastage and, for certain types of material, may require multiple additional processing steps to ensure that the component is suitable for use in a wound dressing, for example sealing and or smoothing cut edges.

Topical negative pressure therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds and abdominal wounds or the like. Wound dressings used in such therapy can require specific components or layers to facilitate the application of reduced pressure.

One such component is a spacer layer which provides a fluid path for gaseous flow, allowing negative pressure to be applied and supporting the transport of wound exudate. The spacer layer material can pose manufacturing issues. Due to the stretch properties and poor dimensional stability of the spacer layers, it can be challenging handling these materials when processed as a rolled goods as they are difficult to control in roll to roll processes. Further, for these reasons, any inline cutting or slitting is also difficult to control.

One method for attempting to circumvent this problem is to use a batch process rather than a continuous process. However, batch processing also has limitations as it is slow, highly labour intensive and abrasive to the cutting blades. It also results in a high amount of fibre shedding, so poses a health and safety hazard which requires extraction, such as air extraction, to control.

It is an aim of the present invention to solve some of the above referenced technical problems.

### SUMMARY

According to one example, there is provided a method of manufacturing a fabric for a wound dressing, the method comprising providing a knitting machine configured to knit a plurality of yarns to form a knitted fabric, the knitting machine comprising a first needle bed comprising a first set of adjacent needles and a second set of adjacent needles; knitting a first fabric by providing a first set of yarns to the first set of adjacent needles; knitting a second fabric by providing a second set of yarns to the second set of adjacent needles; wherein the first set of adjacent needles is separated from the second set of adjacent needles by one or more empty needles in the first needle bed. The empty needles are not provided with yarn. The method provides a continuous process for producing fabrics with a desired width. This method also avoids the requirement of using a batch process, which is generally slow, highly labour intensive and is abrasive to the cutting blades. Further, employing a continuous process helps to reduce the potential for fibre shedding and can reduce the requirement for extraction.

The fabric may comprise a spacer fabric.

The knitting machine may comprise a second needle bed. In one example, the second needle bed comprises a first set of adjacent needles and a second set of adjacent needles, wherein the first set of adjacent needles is separated from the second set of adjacent needles by one or more empty needles in the second needle bed. In this example, a third yarn may be provided to the first set of adjacent needles in the second needle bed and a fourth yarn may be provided to the second set of adjacent needles in the second needle bed. The first fabric may be knitted by a combination of the first set of adjacent needles in the first needle bed and the first set of adjacent needles in the second needle bed and the second fabric may be knitted by a combination of the second set of adjacent needles in the first needle bed and the second set of adjacent needles in the second needle bed.

According to one example, there is provided a fabric formed according to the method above.

The fabric may comprise a spacer fabric comprising a first layer and a second layer.

One or more of the first layer and the second layer may be absorbent.

In one example, the first and second layers are spaced to define a gap suitable for incorporating an additional component of a wound dressing within the gap.

The fabric may include one or more monofilament fibres that are configured to extend between the first layer and the second layer.

The fabric may be for use in a negative pressure wound therapy dressing.

According to one example, there is provided a system comprising a knitting machine comprising a needle bed comprising a plurality of needles; a first set of yarns coupled with a first set of adjacent needles in the needle bed; and a second set of yarns coupled with a second set of adjacent needles in the needle bed, wherein the first set of needles is separated from the second set of needles by one or more empty needles in the first needle bed.

In one example, the needle bed comprises a second needle bed comprising a first set of adjacent needles and a second set of adjacent needles, wherein the first set of adjacent needles is separated from the second set of adjacent needles by one or more empty needles in the second needle bed. In this example, a third yarn may be provided to the first set of adjacent needles in the second needle bed and a fourth yarn may be provided to the second set of adjacent needles in the second needle bed. The first fabric may be knitted by a combination of the first set of adjacent needles in the first needle bed and the first set of adjacent needles in the second needle bed and the second fabric may be knitted by a combination of the second set of adjacent needles in the first needle bed and the second set of adjacent needles in the second needle bed.

According to one example, there is provided a computer program product comprising instructions to cause the system outlined above to execute the steps of the method.

According to one example, there is provided a non-transitory computer readable medium having stored thereon the computer program.

### BRIEF DESCRIPTION OF DRAWINGS

Embodiments of the present disclosure will now be described hereinafter, by way of example only, with reference to the accompanying drawings in which:

FIG. 1 illustrates a cross-section through an example of a wound dressing including a spacer layer;

FIG. 2A illustrates an example of a warp knitting pattern;

FIG. 2B illustrates an example of a weft knitting pattern;

FIG. 3 illustrates an example of a spacer fabric;

FIG. 4 shows an example of part of a knitting machine for producing a spacer fabric;

FIG. 5A shows a schematic example of a knitting machine with a knitting bed for producing a fabric;

FIG. 5B shows a schematic example of a knitting machine with a knitting bed for producing a fabric; and

FIG. 6 shows an example of method steps for producing a fabric.

### DESCRIPTION

Embodiments disclosed herein relate to apparatuses and methods of manufacturing a fabric for a wound dressing. The wound dressing may be used for treating a wound with or without reduced pressure, including for example a source of negative pressure and wound dressing components and apparatuses. The apparatuses and components comprising the wound overlay and packing materials or internal layers, if any, are sometimes collectively referred to herein as dressings. In some embodiments, the wound dressing can be provided to be utilized without reduced pressure.

Some embodiments disclosed herein relate to wound therapy for a human or animal body. Therefore, any reference to a wound herein can refer to a wound on a human or animal body, and any reference to a body herein can refer to a human or animal body.

The disclosed technology may relate to preventing or minimising damage to physiological tissue or living tissue, or to the treatment of damaged tissue e.g., a wound as described below.

As used herein the expression “wound” may include any injury to living tissue and may be caused by a cut, blow, or other impact, typically one in which the skin is cut or broken. A wound may be a chronic or acute injury. Acute wounds occur as a result of surgery or trauma. They move through the stages of healing within a predicted timeframe. Chronic wounds typically begin as acute wounds. The acute wound becomes a chronic wound when it does not follow the healing stages resulting in a lengthened recovery. It is believed that the transition from acute to chronic wound can be due to a patient being immuno-compromised.

Chronic wounds may include for example: Venous ulcers: Venous ulcers usually occur in the legs, account for the majority of chronic wounds, and mostly affect the elderly, Diabetic ulcers (typically foot or ankle ulcers, Peripheral Arterial Disease, Pressure ulcers, or Epidermolysis Bullosa (EB).

Examples of other wounds include, but are not limited to, abdominal wounds or other large or incisional wounds, either as a result of surgery, trauma, sternotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

The wound may also include a deep tissue injury. The deep tissue injury is a term proposed by the National Pressure Ulcer Advisory Panel (NPUAP) to describe a unique form of pressure ulcers. These ulcers have been described by clinicians for many years with terms such as purple pressure ulcers, ulcers that are likely to deteriorate and bruises on bony prominences.

The wound may also include tissue at risk of becoming a wound as discussed above. For example, tissue at risk may include tissue over a bony protuberance (at risk of deep tissue injury/insult), pre-surgical tissue (e.g. knee) that may have the potential to be cut (for joint replacement/surgical alteration/reconstruction).

In some embodiments the disclosed technology relates to a method of treating a wound with the technology disclosed herein in conjunction with one or more of the following: advanced footwear, turning a patient, offloading examples such as diabetic foot ulcers, treatment of infection, systemic, antimicrobial, antibiotics, surgery, removal of tissue, affect blood flow, physiotherapy, exercise, bathing, nutrition, hydration, nerve stimulation, ultrasound, electrostimulation, oxygen therapy, microwave therapy, active agents ozone, antibiotics, antimicrobials, and the like.

The wound may be treated using topical negative pressure and/or traditional advanced wound care i.e., not aided by the using of applied negative pressure (may also be referred to as non-negative pressure therapy).

Advanced wound care may include use of an absorbent dressing, an occlusive dressing, use of an antimicrobial and/or debriding agents in a wound dressing or adjunct, a pad e.g., a cushioning or compressive therapy (such as stocking or bandages).

In some embodiments treatment of such wounds can be performed using traditional wound care, wherein a dressing can be applied to the wound to facilitate and promote healing of the wound.

The wound dressings that may be utilized in conjunction with the disclosed technology include any known dressing in the art. The technology is applicable to negative pressure therapy treatment as well as non-negative pressure therapy treatment.

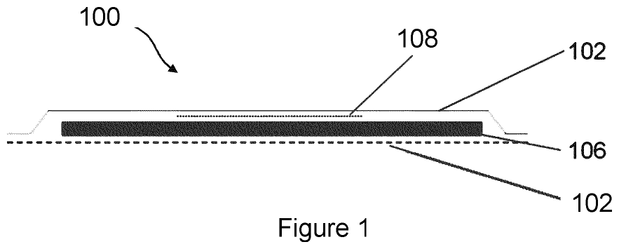

FIG. 1 shows an example of a cross-section through a wound dressing 100.

In one example, the wound dressing 100 comprises a spacer layer 106, which in this case is a three-dimensional spacer fabric. The wound dressing 100 may include an absorbent layer or layers in addition to the spacer layer 106. Details of the formation of the spacer layer 106 is provided below.

The wound dressing 100 may also include a top film 102 and wound contact layer 104, which may be perforated. In one example, the spacer layer 106 may be contained between the wound contact layer 104 and the top film 102.

The wound contact layer 104 may comprise a perforated wound-side adhesive which can be a silicone adhesive, or a low-tack adhesive to minimise skin trauma on removal. The wound contact layer 104 may comprise a support material which can be a mesh, a net or a perforated film. The wound contact layer 104 may also comprise a construction adhesive on the pad side, to ensure its intimate contact with the lowest part of the pad, and therefore efficient uptake of fluid from the wound without pooling.

The top film 102 may be a liquid-impermeable, moisture-vapour permeable, breathable film, which allows moisture to evaporate from the dressing. The top film 102 may be considered to be a cover layer for covering the other layers of the wound dressing 100, helping to encapsulate the layers between the wound contact layer 102 and the top film 102. In one example, the top film 102 is a layer of polyurethane, Elastollan (trade name) SP9109 manufactured by BASF. The top film 102 may be coated with any suitable adhesive. Aptly the adhesive will be a pressure sensitive adhesive e.g. acrylic adhesive or silicone adhesive.

The wound dressing 100 layer may include a masking layer 108 to allow partial masking of the top surface of the spacer layer 106, where coloured exudate could remain.

It will be understood that embodiments of the present disclosure are generally applicable to use in Topical Negative Pressure (TNP) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

The spacer layer 106 has a three-dimensional knitted structure. One function of the spacer layer 106 may be for pressure distribution and impact protection. For example, if the patient accidentally knocks the wound area, leans on the wound area or another cause applies a pressure to the dressing covering a wound. Aptly the spacer layer 106 may be provided closer to where the pressure is being applied than other layers of the dressing.

Further, the spacer layer 106 provides a fluid path in the wound dressing 100. In one example, the fluid path enables a negative pressure to be delivered to the wound, as will be discussed in more detail below.

Knitting is one of several ways to turn yarns into fabric. Knitted fabric may include horizontal parallel courses of yarn. The courses are joined to each other by interlocking loops in which a short loop of one course of yarn is wrapped over a bight of another course of yarn. Knitting can be done either by hand or by using a knitting machine, such as a “Karl Mayer spacer machine RD6N”.

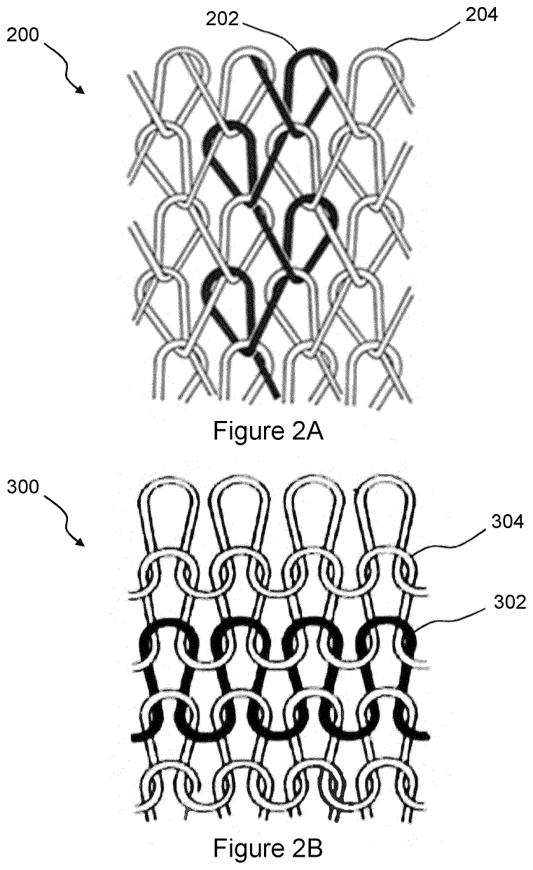

FIG. 2A shows an example of a Warp knitted fabric 200. Traditional warp knitting is the sequential formation and interlinking of loops in an axial direction on a needle bed. The loops are joined together in a width-wise direction by moving the threads back and forth between adjacent needles by using a guide bar. In the example shown in FIG. 2A, a first yarn 202 is shaded to illustrate the path of the first yarn in the warp knit structure. In this example, the first yarn 202 was provided to a first needle. A second yarn 204 was provided to a second, adjacent, needle. In warp knitting, the needles produce parallel rows of loops simultaneously that are interlocked in a zigzag pattern, as shown in FIG. 2A.

The warp knitting process consists of mainly two parts; the stitch and the lap. The stitch is formed by wrapping the yarn around the needle and drawing it through the previously knitted loop. This wrapping of the yarn is called an overlap. The second part of stitch formation is the length of yarn linking together the stitches and this is termed the underlap, which is formed by the lateral movement of the yarns across the needles.

A weft knitted structure is shown in FIG. 2B. Weft knitting is a method of forming a fabric in which the loops are formed in a horizontal manner from a yarn and intermeshing of loops take place in a circular or flat form on a crosswise basis. In this method each weft yarn is fed substantially perpendicularly to the direction in which fabric is formed. Each course in a weft knit builds upon the previous knitted course. In the example shown in FIG. 2B, a first yarn 302 is shaded to illustrate the path of the first yarn 302 in the weft knit structure. In this example, the first yarn 202 was provided to a first needle. A second yarn 304 was provided to a second needle. In weft knitting, the needles produce parallel rows of loops simultaneously that are interlocked in a zigzag pattern, as shown in FIG. 2B.

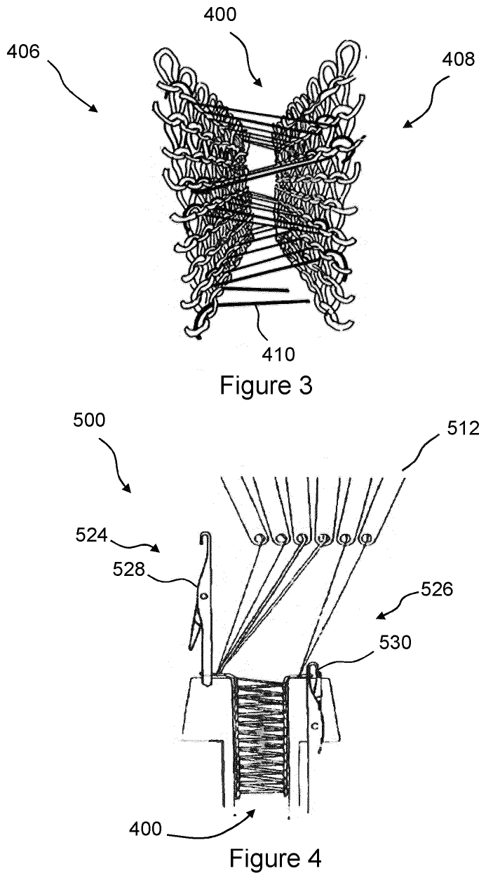

Knitted spacer fabrics 400 are structures that consist of two separately produced fabric layers 406, 408 which are joined traditionally by spacer yarns, such as monofilament yarns. An example of a knitted spacer fabric 400 is shown in FIG. 3. The two layers 406, 408 may be produced from different materials and may have completely different structures. Spacer yarns 410 which join the two layers 406, 408 can either fix the layers 406, 408 directly or space them apart. It is this three-dimensional space which is the special feature of these structures. In one example, the spacer yarns 410 comprise monofilament yarns.

Knitting machines may be equipped with a needle-bed comprising rows of small, hooked needles to draw formed yarn loops through previously formed loops.

Knitting machines may be equipped with one or more needle-beds 524, 526. In one example, the needle-bed of a knitting machine is substantially flat, but in other examples, the needle bed may have a substantially circular profile on plan. The needle-bed may be made up of a plurality of needles and a steel body provided with grooves where the needles with hook and butt turned upward slide. The grooves guide the needles during the knitting process. FIG. 4 shows an example of a cross-sectional view of part of a knitting machine 500 for forming a spacer fabric 400, as shown in FIG. 3. The example of a knitting machine 500 shown in FIG. 4 includes a first needle bed 524 and a second needle bed 526. Only a single needle 528 from the first needle bed 524 is visible in FIG. 4. Further, only a single needle 530 from the second needle bed 526 is shown. The knitting machine 500 shown in FIG. 4 is able to produce two face layers 406, 408 simultaneously using the first needle bed 524 and the second needle bed 526. The two face layers 406, 408 layers are connected by monofilament yarns (or spacer yarns) 410 which during production are transferred from the first needle bed 524 to the second needle bed 526 and back again in a systematic method of joining the two face layers 406, 408 of the spacer yarn 400.

The knitting machine 500 may comprise a plurality of guide bars 512. In this example, the two left-hand side guide bars 512 are configured to move the yarn around the needles in the first needle bed 524, the two right-hand side bars 512 are configured to move the yarn around the needles in the second needle bed 526. The two middle guide bars shown in FIG. 4 are configured to carry the spacer yarn 410 and knit on both needle beds 524, 526 in succession.

Each of the first needle bed 524 and the second needle bed 526 includes a plurality of needles 528, 530. The needle 528, 530 is the basic element of loop formation. Needles used in knitting machines can be a latch needle, a spring-beard needle and/or a compound needle. Characteristics of these needles may differ, but the basic components of the needle are a hook, stem, butt. Needles are known in the art and so further details of needles have not been included here.

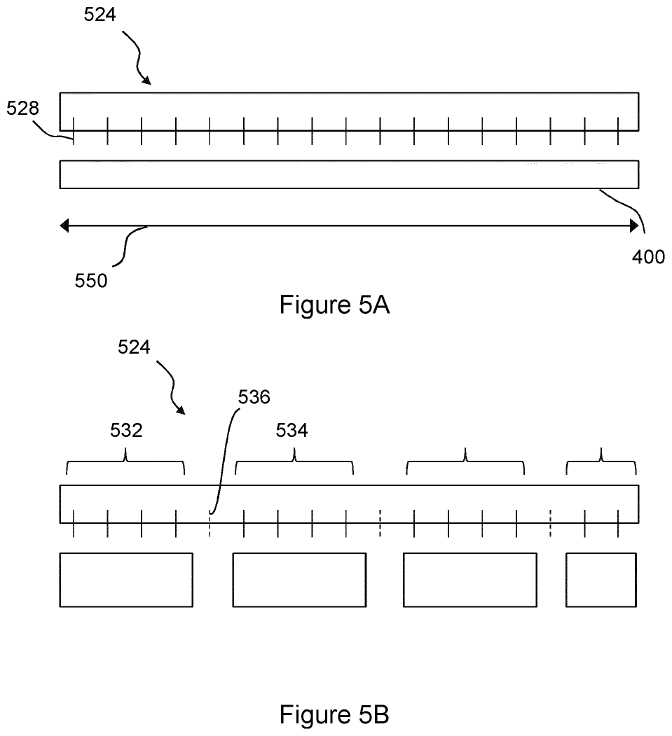

A schematic example of the first needle bed 524 comprising a plurality of needles 528 is shown in FIG. 5A. The second needle bed 526 is substantially identical to the first needle bed and so FIG. 5A may also be considered to show a schematic example of the second needle bed 526. In this example, each of the needles 528 is provided with a yarn and the needle bed 524 knits the fabric across the full length of the needle bed, which produces a fabric with width (w) 550. Typical needle beds 524 have a width of between 1.5 m and 5 m.

FIG. 5B shows the same the needle bed 524 comprising a plurality of needles 528 as shown in FIG. 5A. However, in this example, the needles 524 comprise a first set of adjacent needles 532 and a second set of adjacent needles 534. By adjacent needles, it is meant that there is a continuous set of needles. In other words, the first set of adjacent needles 532 comprises an uninterrupted set of needles without another needle located therebetween. Further, the second set of adjacent needles 534 comprises an uninterrupted set of needles without another needle located therebetween.

In the example shown in FIG. 5B, a first set of yarns is provided to the first set of adjacent needles 532 in the needle bed 524 and a second set of yarns is provided to the second set of adjacent needles 534 in the needle bed 524. The first set of adjacent needles 532 is separated from the second set of adjacent needles 534 by one or more empty needles 536 that are not provided with yarn. In other words, the first set of needles 532 is spaced apart from the second set of needles 534 by one or more needles 536 that will not contribute to the knitting process. Providing the separation between the first set of needles 532 and the second set of needles 534 means that the knitting machine is able to produce a first fabric using the first set of needles and a second fabric, not connected to the first fabric, using the second set of needles. The method of production means that multiple fabrics can be produced simultaneously in a continuous process and at a desired width, that need not be the whole width of the needle bed 524. The first set of needles 532 may comprise a different number of needles compared to the second set of needles 534. In one example, the first set of needles 532 comprises between 200 and 1000 needles. However, in practice, there may be fewer or more needles in the first set of needles 532. In one example, the second set of needles 532 comprises between 200 and 1000 needles. However, in practice, there may be fewer or more needles in the second set of needles 534. In one example, only a single empty needle 536 is located between the first set of needles 532 and the second set of needles 534, but in other examples more than one empty needles 536 is located between the between the first set of needles 532 and the second set of needles 534. Providing multiple empty needles helps the draw out and consequent rolling of the goods as they come out of the knitting machine. Providing wider gaps in between pieces gives a greater tolerance on winding which would be helpful.

This method may be used to knit thinner strips of fabric, eliminating the need of a slitting process. This process may also be used to produce spacer fabric 400 to alter the placement of the spacer yarns 410, which can be used to create areas in the spacer fabric 400 with no spacer yarns 410 between face layers 406, 408. The removal of these spacer yarns 410 would increase ease of cutting and less force would be required to cur the spacer fabric 400. Further, no loose waster spacer yarns 410 would be created, so the requirements for control of these loose filaments would be eliminated. As such, this process makes it possible to manufacture a rolled good in the desired width of dressing, with intervals where no spacer yarn 410 present. These intervals correspond to the positions of the needles 536 to which no yarn was provided.

In one example, the method outlined above is used to produce a plurality of warp-knitted fabrics 200 from the first set of needles 532 and the second set of needles 534 respectively. In other examples, the method outlined above is used to produce a plurality of weft-knitted fabrics 300 from the first set of needles 532 and the second set of needles 534 respectively. In one example, the method outlined above is used to produce a spacer fabric 400.

Depending on the position of the one or more empty needles 536 to which no yarn is provided, the spacer fabric 400 may be produced with designated areas in which there is no spacer yarn 410. Alternatively, the machine may produce a plurality of spacer fabrics 400 simultaneously. These spacer fabrics 400 would have a shorter width compared with the width of the spacer fabric that would be produced without using the empty needle process.

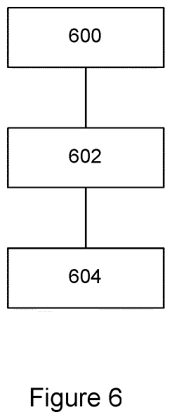

FIG. 6 shows an example of the method steps for manufacturing a fabric for a wound dressing. As step 600, the method comprises the step of providing a knitting machine 500 configured to knit a plurality of yarns to form a knitted fabric, the knitting machine comprising a needle bed 524 comprising a first set of adjacent needles 532 and a second set of adjacent needles 534.

At step 602, the method comprises the step of providing a first set of yarns to the first set of adjacent needles 532.

At step 604, the method comprises the step of providing a second set of yarns to the second set of adjacent needles 534. The first set of adjacent needles 532 is separated from the second set of adjacent needles 534 by one or more empty needles 536. The empty needles 536 are needles that are not provided with yarn.

In use, a wound dressing as described above would be applied to a wound site of a patient with the surface of the substrate according to the invention facing the wound site. Any wound exudate, blood or other wound fluid would travel into the dressing via the substrate according to the invention and sequential layers above the substrate according to the invention. Fluid would permeate through the foam layer, the activated charcoal layer, and then reach the absorber layer at which point preferably the liquid would not go any further and be retained by the absorber layer. On the other hand, gas and moisture vapour would be able to permeate further via the spacer layer and/or top film.

The wound facing surface of a wound dressing may be provided with a release coated protector (not shown in the figures), for example a silicon-coated paper. The protector covers the wound contacting side of the dressing prior to application to a patient and can be peeled away at the time of use.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. A method of manufacturing a fabric for a wound dressing, the method comprising:
providing a knitting machine configured to knit a plurality of yarns to form a knitted fabric, the knitting machine comprising a first needle bed comprising a first set of adjacent needles and a second set of adjacent needles;
knitting a first fabric by providing a first set of yarns to the first set of adjacent needles;
knitting a second fabric by providing a second set of yarns to the second set of adjacent needles;
wherein the first set of adjacent needles is separated from the second set of adjacent needles by one or more empty needles in the first needle bed.

2. The method of claim 1, wherein the fabric comprises a spacer fabric.

3. The method of claim 1, wherein the knitting machine comprises a second needle bed comprising a first set of adjacent needles and a second set of adjacent needles, wherein the first set of adjacent needles is separated from the second set of adjacent needles by one or more empty needles in the second needle bed.

4. A fabric formed according to the method of claim 1.

5. The fabric according to claim 4, wherein the fabric comprises a spacer fabric comprising a first layer and a second layer.

6. The fabric according to claim 5, wherein one or more of the first layer or the second layer is absorbent.

7. The fabric according to claim 5, wherein the first layer and the second layer are spaced to define a gap suitable for incorporating an additional component of a wound dressing within the gap.

8. The fabric according to claim 6, further comprising one or more monofilament fibres that are configured to extend between the first layer and the second layer.

9. The fabric according to claim 5, wherein the fabric is for use in a negative pressure wound dressing.

10. A system comprising:
a knitting machine comprising a first needle bed comprising a plurality of needles;
a first set of yarns coupled with a first set of adjacent needles in the first needle bed; and
a second set of yarns coupled with a second set of adjacent needles in the first needle bed,
wherein the first set of needles is separated from the second set of needles by one or more empty needles in the first needle bed.

11. The system according to claim 10, wherein the knitting machine further comprises a second needle bed comprising a first set of adjacent needles and a second set of adjacent needles, wherein the first set of adjacent needles is separated from the second set of adjacent needles by one or more empty needles in the second needle bed.

12. A computer program product comprising instructions to cause the system of claim 10 to execute the steps of the method of claim 1.

13. A non-transitory computer readable medium having stored thereon the computer program product of claim 12.

14. A wound dressing comprising a spacer fabric, the spacer fabric comprising:
a first layer comprising a first knitted fabric and a second layer comprising a second knitted fabric,
wherein the first and second knitted fabrics are formed with a predetermined width suitable for direct incorporation in the wound dressing without further processing.

15. The wound dressing of claim 14, wherein at least one of the first layer or the second layer is absorbent.

16. The wound dressing of claim 14, wherein the first layer and the second layer are spaced to define a gap suitable for incorporating an additional component of the wound dressing within the gap.

17. The wound dressing of claim 14, further comprising one or more monofilament fibres extending between the first layer and the second layer of the spacer fabric and not extending beyond an edge of the spacer fabric.

18. The wound dressing of claim 14, wherein the spacer fabric is configured to distribute pressure applied to a top of the wound dressing.

19. The wound dressing of claim 14, wherein the spacer fabric has no cut edges.

20. The wound dressing of claim 14, wherein the first knitted fabric and the second knitted fabric are made by:
providing a knitting machine configured to knit a plurality of yarns to form a knitted fabric, the knitting machine comprising a first needle bed comprising a first set of adjacent needles and a second set of adjacent needles;
knitting the first knitted fabric by providing a first set of yarns to the first set of adjacent needles; and
knitting the second knitted fabric by providing a second set of yarns to the second set of adjacent needles, wherein the first set of adjacent needles is separated from the second set of adjacent needles by one or more empty needles in the first needle bed.
